(19) United States
(12) Patent Application Publication
(10) Pub. No.: US 2023/0392181 A1
(43) Pub. Date: Dec. 7, 2023

(54) METHODS FOR DETECTING ZEROVALENT NANOPARTICLE-MEDIATED OXIDATIVE STRESS IN CYANOBACTERIA

(71) Applicant: Morgan State University, Baltimore, MD (US)

(72) Inventors: Viji Sitther, Baltimore, MD (US); Samson Gichuki, Nottingham, MD (US)

(21) Appl. No.: 18/329,130

(22) Filed: Jun. 5, 2023

Related U.S. Application Data
(60) Provisional application No. 63/348,597, filed on Jun. 3, 2022.

(51) Int. Cl. C12Q 1/02; G01N 33/573

(52) U.S. Cl. CPC C12Q 1/025; G01N 33/573; G01N 2333/90283

(57) ABSTRACT

Zero-valent iron nanoparticle-mediated oxidative stress was evaluated in F. diplosiphon as a measure of lipid peroxidation using malondialdehyde and thiobarbituric acid-reactivity (MDA-TBA) assay and the 2′,7′-dichlorodihydrofluorescein diacetate (DCFH-DA) fluorogenic probe. Enzymatic response of nZVI-treated cultures was determined as a measure of FeSOD (iron superoxide dismutase) using western blotting and immunodetection. In addition, the distribution and surface assimilation of nZVIs was detected using transmission electron microscopy (TEM) and energy-dispersive X-ray spectroscopy (EDS).

### FIELD OF THE INVENTION

The invention relates to the detection of nZVI-mediated oxidative stress in cyanobacteria.

### BACKGROUND OF THE INVENTION

As an important element for most living organisms, iron serves as a cofactor in a multitude of cellular processes such as respiration, photosynthesis, and nitrogen fixation. In phototrophs such as cyanobacteria, iron is indispensable for redox-related functions in the photosynthetic apparatus.¹ Cyanobacterial iron requirements exceed that of non-photosynthetic prokaryotes by multiple folds and are exceptionally high compared to other photosynthetic organisms.² It is well known that in cyanobacteria, iron homeostasis is tightly controlled by the ferric uptake regulator which modulates the transcription of iron-responsive genes.¹⁶ Present in the form of ferredoxins (Fds) within the thylakoid membrane, iron mediates electron transfer in metabolic reactions and facilitates the reduction of NADP+ to NADPH by carrying an electron from the iron-sulfur complex to the Fd-NADP+ reductase.³ However, due to its redox properties, iron has the capacity to react with oxygen, thus generating reactive oxygen species (ROS) resulting in the rapid generation of free radicals. Redox-active Fe0 reacts with oxygen or water and releases Fe2+ that further generates ROS via the Fenton chemistry.⁴

In recent years, cultivation of various algal and cyanobacterial strains with nanoscale zero-valent iron nanoparticles (nZVIs) has been reported to boost growth and lipid production.^{5,6} The relatively low standard reduction potential enables nZVIs to act as electron donors, catalyzing a wide range of reactions.⁷ Of the various cyanobacterial species, Fremyella diplosiphon is a widely studied model organism with increased fitness to grow in a broad spectrum of light. Additionally, the organism grows in a short generation time of 7-10 days, making it an ideal model for several studies.⁸ In recent years, metallic nanoparticles have been shown to boost growth, photosynthetic pigmentation, and the fatty acids accumulated in this organism, making it an attractive third-generation biofuel agent.^{9,10}

While nanoparticles are known to positively impact cellular processes in cyanobacteria, oxidative stress induced by an imbalance of intracellular ROS is inevitable. Disruption of cellular organelles and physiological processes due to excessive ROS in cyanobacteria has been reported.¹¹ Specifically, singlet oxygen is a highly reactive ROS species that targets molecules such as proteins, pigments, and lipids in the immediate vicinity. In addition, production of ROS increases the intensity of light required for electron transport, which outpaces the rate of electron consumption during carbon assimilation.¹² Several critical enzymes such as superoxide dismutases (SODs), catalases, and peroxidases in this pathway have been reported to be indicators of free radical production and oxidative stress in cyanobacterial cells.¹³

Nanoscale zero-valent iron nanoparticles (nZVIs) are known to boost biomass production and lipid yield in Fremyella diplosiphon, a model biodiesel-producing cyanobacterium. However, the impact of nZVI-induced reactive oxygen species (ROS) in F. diplosiphon has not been evaluated.

### SUMMARY OF THE INVENTION

ROS in F. diplosiphon strains (B481-WT and B481-SD) generated in response to nZVI-induced oxidative stress were quantified and the enzymatic response determined. Lipid peroxidation as a measure of oxidative stress revealed significantly higher malondialdehyde content (p<0.01) in both strains treated with 3.2, 12.8, and 51.2 mg L⁻¹ nZVIs compared to untreated control. In addition, ROS in all nZVI-treated cultures treated with 1.6-25.6 mg L⁻¹ nZVIs was significantly higher than the untreated control as determined by the 2′,7′-dichlorodihydrofluorescein diacetate fluorometric probe. Immunodetection using densitometric analysis of iron superoxide dismutase (SOD) revealed significantly higher SOD levels in both strains treated with nZVIs at 51.2 mg L⁻¹. In addition, we observed significantly higher (p<0.001) SOD levels in the B481-SD strain treated with 6.4 mg L⁻¹ nZVIs compared to 3.2 mg L⁻¹ nZVIs. Validation using transmission electron microscopy equipped with energy-dispersive X-ray spectroscopy (EDS) revealed adsorption of nZVIs with a strong iron peak in both B481-WT and B481-SD strains. While the EDS spectra showed strong signals for iron at 4 and 12 days after treatment, a significant decrease in peak intensity was observed after 20 days.

A variety of enzymes such as catalases, SODs, and peroxidases are deployed against excessive ROS generation in cyanobacteria.¹⁸ Of these, malondialdehyde formed due to the breakdown of lipid peroxidation is an important marker that reflects cellular oxidative damage due to nanoparticles.¹⁹ The MDA content in both B481-WT and B481-SD strains exposed to nZVIs from 3.2 to 51.2 mg L⁻¹ was observed to be significantly higher than the control (FIG. 1). It is possible that higher nZVI concentrations could have disrupted the redox stability between production and scavenging of ROS in F. diplosiphon, resulting in the overaccumulation of ROS, thus triggering the peroxidation of membrane lipids. Interestingly, we observed significant differences in the MDA content between B481-WT and B481-SD strains which could be attributed to the fact that the B481-SD strain was overexpressed for higher lipid content.¹⁰ There was no significant difference in the MDA content in cultures treated with 102.4 mg L⁻¹ nZVIs and the untreated control. nZVIs at a high concentration of 102.4 mg L⁻¹ likely induced significant oxidative stress, thus limiting culture growth as confirmed by reduced optical density on day 15. Since lipid peroxidation is directly related to MDA content, a reduction in the MDA content directly correlates to lower growth.

Using the oxidant-sensing DCFH-DA probe which is converted into the highly fluorescent form DCF in the presence of ROS, we observed significantly higher fluorescence in both F. diplosiphon strains treated with nZVIs ranging from 3.2 to 25.6 mg L⁻¹, indicating higher levels of ROS with the increase in nZVI concentrations (FIG. 2). Interestingly, we observed lower levels of fluorescence in the B481-SD strain compared to B481-WT in all nZVI concentrations tested.

Although various types of SODs such as Fe-SOD, Mn-SOD, and Ni-SOD are expressed in response to stress tolerance in prokaryotes,²² we tested FeSOD activity due to its affinity, selectivity, and its importance as the most ancient type of SOD crucial for cell survival.²³ Our results revealed that both B481-WT and B481-SD strains treated with 51.2 mg L⁻¹ nZVIs exhibited significantly increased SOD levels (FIG. 3), indicating that the cells suffered from sustained oxidative stress. It is known that nZVIs tend to release electrons and iron (II) ions, which convert less reactive hydrogen peroxide into more ROS via the Fenton reaction.¹¹ Iron is essential for optimal FeSOD activity, as reported in Spirulina platensis exposed to several environmental stresses including iron, pH, and salinity. A broad variation in specific SOD activities has been observed in different cyanobacterial species, with maximal SOD activity of Anabaena variabilis recorded at only 0.02 mM Fe, while in S. maxima, it was at 0.036 mM iron.²⁴ It is possible that the nZVI-mediated oxidative stress could have elevated the level of signal transduction, leading to an activation of existing enzyme pools or enhanced expression of FeSOD gene transcription and enzymatic activity, particularly at concentrations of 51.2 mg L⁻¹ nZVIs.

While TEM imaging provided morphological information on the adsorption of nZVIs to F. diplosiphon cells, EDS revealed qualitative analysis, distribution, and elemental mapping. A strong iron peak in the EDS spectra (FIG. 4A) confirmed the presence of iron in F. diplosiphon-treated cultures. Our results on spectral analysis indicate that the nZVIs were progressively uptaken by F. diplosiphon B481-WT and B481-SD strains. The variation in nZVI sizes observed (FIG. 4B) could be attributed to the magnetic nature and high surface area to volume ratio of the iron nanoparticles, which could have resulted in agglomeration.^{4,26} Although EDS is an analytical technique used to determine the chemical elements present in a sample, quantitative analysis could be affected by several factors. While the EDS spectra of nZVI-treated strains exhibited strong signals for iron after day 4 and day 12 (FIGS. 5a, 5b), a significant decrease in the iron peak intensity was observed after 20 days (FIG. 5c). Cellular uptake of the nZVIs on day 4 was lower and the strong Fe signal was mostly from iron nanoparticles adsorbed onto the surface of the cells. After day 12, it is possible that most of the nZVIs dissolve and get absorbed into the cells, leading to a decrease of the iron signal detected by the EDS.²⁷ It is likely that most nanoparticles were internalized by day 20 and the electron beam was not able to sufficiently interact with the nZVIs, resulting in a lower intensity of the iron peak.

### BRIEF DESCRIPTION OF DRAWINGS

FIG. 1. Quantification of malondialdehyde as a measure of lipid peroxidation in F. diplosiphon B481-WT and B481-SD strains treated with nZVIs at concentrations ranging from 0.8 to 102.4 mg L⁻¹. Average MDA concentration (±standard error) of three biological replicates for each strain is shown. Different letters above the final concentration point indicate significance among treatments (p<0.01).

FIG. 2. Intracellular reactive oxygen species validated using highly fluorescent DCF in F. diplosiphon B481-WT and B481-SD strains grown in BG-11/HEPES medium supplemented with 1.6, 3.2, 6.4, 12.8, and 25.6 mg L⁻¹ nZVIs. Average DCF fluorescence (±standard error) of three biological replicates for each sample is shown. Different letters above the error bars indicate significance among treatments (p<0.01).

FIG. 3. Non-denaturing PAGE analysis of FeSOD in F. diplosiphon B481-WT and B481-SD strains grown in BG-11/HEPES medium containing 3.2, 6.4, and 51.2 mg L⁻¹ nZVIs. Different letters above the final concentration point indicate significance among treatments (p<0.05).

FIG. 4A. EDS spectrum of bare nZVIs nanoparticles.

FIG. 4B. Histogram showing size distribution of nZVIs used in the present study.

FIG. 5A. EDS of F. diplosiphon treated with 3.2 mg L⁻¹ nZVIs after exposure to nZVIs on day 4

FIG. 5B, EDS of F. diplosiphon treated with 3.2 mg L⁻¹ nZVIs after exposure to nZVIs on day 12.

FIG. 5C. EDS of F. diplosiphon treated with 3.2 mg L⁻¹ nZVIs after exposure to nZVIs on day 20.

### DETAILED DESCRIPTION OF THE INVENTION

The invention summarized above may be better understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

### Experimental Section

Strains and Culture Conditions. F. diplosiphon strains, B481-WT obtained from the UTEX algal repository (Austin, TX, USA) and B481-SD (overexpressed strain with the sterol desaturase gene; accession: MH329183), were used in this study. The strains were grown in liquid BG-11 medium containing 20 mM HEPES (hereafter termed as BG-11/HEPES) to an exponential growth phase (˜optical density 750 nm of 0.8). Cultures were grown in vented 40 mL tissue culture flasks with continuous shaking at 70 rpm and 28° C. in an Innova 44R incubator shaker (Eppendorf, Hamburg, Germany). The spectrum of photosynthetic light in the shaker had peak wavelengths at 437 and 600-650 nm with an intensity adjusted to 30 μmol m−2 s⁻¹ using the model LI-190SA quantum sensor (Li-Cor, Lincoln, NE, USA). Nanofer 25 s zero-valent iron nanoparticles (nZVIs) coated with polyacrylic acid were obtained from Nano iron company (Rajhrad, Czech Republic) and adjusted to final concentrations of 1.6, 3.2, 6.4, 12.8, 25.6, 51.2, and 102.4 mg L⁻¹.

Nanoscale Zero-Valent Nanoparticle-Induced Lipid Peroxidation in F. diplosiphon. Quantification of oxidative stress in F. diplosiphon strains treated with nZVIs was performed using the thiobarbituric acid-malondialdehyde (TBA-MDA) adduct formation according to the method described by Bhandari and Sharma²⁸ with modifications. Cultures were grown in BG-11 media containing nZVIs at concentrations of 1.6, 3.2, 6.4, 12.8, 51.2, and 102.4 mg L⁻¹ for a period of 15 days under culture conditions mentioned above. Cultures not treated with nZVIs served as the control. Three replicates were maintained for each treatment, and the experiment was repeated once. A total of 5 mL cells from each treatment was centrifuged at 18,000 rpm for 15 min at 4° C. The pellet was weighed and homogenized in 1 mL of 0.5% trichloroacetic acid (TCA), brought to 5 mL final volume with TCA, and centrifuged at 14,000 rpm for 15 min at 4° C. To a test tube containing 2.5 mL of 0.5% TBA in 20% TCA, 1 mL of the supernatant was added and incubated for 30 min at 90° C. in a water bath. The mixture was centrifuged for 2 min at 3000 rpm to remove the nonspecific precipitate. Absorption was measured at 532-600 nm using a microplate reader (BioTek Synergy H1 Microplate Reader, Agilent, USA). Peroxidation of lipids was calculated using an extinction coefficient of ε=155 mM⁻¹ cm⁻¹.

Detection of Intracellular ROS in Fremyella diplosiphon Treated with nZVIs.

Intracellular ROS in nZVI-treated F. diplosiphon was measured using the 2′,7′-dichlorodihydrofluorescein diacetate (DCFH-DA) fluorometric probe (EMD Chemicals, USA) according to the method described by Pattanaik et al.²⁹ B481-WT and B481-SD strains were grown in nZVIs at 1.6, 3.2, 6.4, 12.8, and 25.6 mg L⁻¹ under culture conditions as mentioned above. As cultures treated with 51.2 mg L⁻¹ nZVIs resulted in elevated oxidative stress as observed in the lipid peroxidation assay, this concentration was not used to detect intracellular ROS.

Three biological replicates were maintained, and the experiment was repeated once. Cultures not treated with nZVIs served as the control. At 15 days after treatment, 1 ml of each culture was incubated in 10 mM DCFH-DA for 1 h at room temperature in the dark and under shaking at 70 rpm. Fluorescence of DCF-DA converted from the nonfluorescent DCFH-DA was detected by loading 200 μl of each sample in a 96 well-plate and read at an excitation wavelength of 485 nm and an emission of 520 nm in a microplate reader (BioTek Synergy H1 Microplate Reader, USA). Three technical replicates on a 96-well plate were measured for each treatment to account for plating bias. A negative control of bare nZVIs was used to account for any potential interference of autofluorescence that may interfere with the DCFH-DA probe signal.

Enzymatic Response of Fremyella diplosiphon to Nanoscale Zero-Valent Nanoparticle-Mediated Oxidative Stress.

Protein Extraction. F. diplosiphon B481-WT and B481-SD strains were grown in BG11 media/HEPES media supplemented with 3.2, 6.4, and 51.2 mg L⁻¹ nZVIs. Cultures were grown in 250 mL conical flasks with continuous shaking at 170 rpm, 28° C., and ambient CO2 (350-1000 mg L⁻¹) in an Innova 44R incubator shaker (Eppendorf, Hamburg, Germany). Cultures not treated with nZVIs served as the control. After 9 days, cells were pelleted at 8000 rpm for 15 min at 4° C. and total proteins extracted. For each sample, two protease inhibitor tablets (Roche) were dissolved in 30 mL of binding buffer containing 50 μM tris and 500 μM NaCl. Lysozyme (0.03 g) was dissolved in 3 mL of binding buffer, added to each sample, and incubated in ice for 1 h. The pellet was sonicated in ice at 100% ampere for 10 s with 1 min intervals, and the process was repeated nine times. The lysate was spun down at 10,000 rpm for 15 min at 4° C. and lyophilized, and the protein concentration of each sample determined using the BCA assay Smith et al.³⁰ Samples were diluted to 2.0 mg mL⁻¹ in SDS boiling buffer with reducing agents and heated in a digital dry bath at 95° C. for 10 min before loading in a SDS gel.

Sodium Dodecyl Sulfate-Polyacrylamide Gel Electrophoresis. SDS slab gel electrophoresis was carried out under reducing conditions according to the method of Laemmli,³¹ as modified by O'Farrell.³² The samples were loaded in 10% acrylamide slab gels (0.75 mm thick) and SDS slab gel electrophoresis was carried out for about 4 h at 15 mA/gel. Myosin (220,000), phosphorylase A (94,000), catalase (60,000), actin (43,000), carbonic anhydrase (29,000), and lysozyme (14,000) were used as molecular weight standards. Following slab gel electrophoresis, the gels for blotting were placed in a transfer buffer (10 mM Caps, pH 11.0, 10% methanol) and transblotted onto PVDF membranes overnight at 200 mA and approximately 100 V/two gels. The blots were stained with Coomassie Brilliant Blue R-250 and scanned with GE Imagescanner III.

Western Blot and Densitometric Analysis. The blots were destained in 100% methanol, rinsed in Tween-20 tris buffer saline (TTBS), and blocked for 2 h in 5% nonfat Dry Milk (NFDM) in TTBS. The blots were then incubated overnight in primary antibody (anti-FeSOD [Agrisera, Cat #AS06125] diluted 1:20,000 in 2% NFDM TTBS) and rinsed for 3×10 min in TTBS. The blots were placed in the secondary antibody (anti-Rabbit IgG-HRP [SeraCare, Cat #5220-0337] diluted 1:20,000 in 2% NFDM TTBS) for 2 h, rinsed as above, treated with enhanced chemiluminescence, and exposed to Kodak BioMax MR X-ray film. Western blot films (K05401 #1-3: Kodak BioMax MR 3-min exposures) were scanned with a laser densitometer (Model PDSI, Molecular Dynamics Inc, Sunnyvale, CA). The scanner was checked for linearity prior to scanning with a calibrated Neutral Density Filter Set (Melles Griot, Irvine, CA). Band volume above background (manual marking) was quantified using Phoretix 1D software (version 11.2) with a Windows 10 compatible computer.

Distribution and Validation of nZVIs in Fremyella diplosiphon. F. diplosiphon strains grown in 0.8 mg L⁻¹ nZVIs for 4, 12, and 20 days were tested for distribution and adsorption of nanoparticles using TEM. Samples were prepared by aliquoting 10 μL onto TEM carbon-coated 200-mesh copper grids and dried overnight at room temperature. The sizes of one hundred nZVIs were measured and their average with associated standard deviation calculated. The TEM was equipped with an EDS detector for spectral and elemental mapping.

TEM images of F. diplosiphon-treated with nZVIs, untreated cells, and bare nanoparticles were acquired using the JEM-1400 PLUS (JEOL USA, Massachusetts, USA) equipped with an energy-dispersive X-ray microanalysis system (Oxford Instruments plc, Abingdon, United Kingdom). The images were viewed using Digital Micrograph software from GATAN (GATAN Inc., CA, USA). In addition, samples were fixed to a glass microscope slide and visualized using a Wolfe DigiVu DVM 9.0 digital microscope (Carolina Biological Supply Co., Burlington, NC, USA), and images were captured using a Moticam digital microscopy camera.

Statistical Analysis. Results were reported as cumulative treatment mean±standard error. Statistical significance was determined using one-way analysis of variance and Tukey's honest significant differences post hoc test at 95% confidence intervals (p<0.05). The single-factor, fixed-effect ANOVA model, Yᵢⱼ=μ+αNi_{+εij}, was used where Y represents a single, distinct parameter (growth rate or ROS content) in nZVI treatment i and biological replicate j. The p represents the overall mean adjusted by effects associated with the nZVI treatment (αN) and εij the experimental error from nZVI treatment i and biological replicate j.

Results

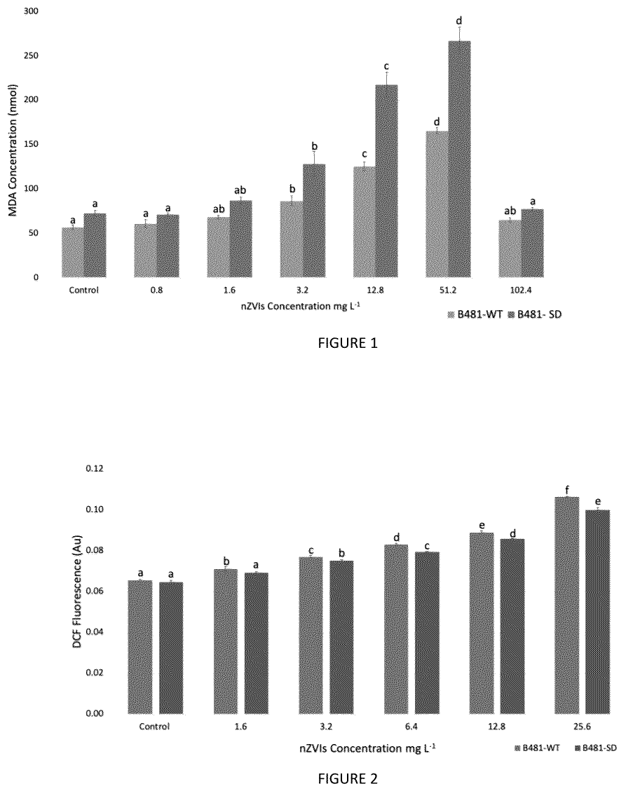

Determination of Lipid Peroxidation as a Measure of ROS. The level of malondialdehyde production, which serves as an indicator for ROS in nZVI-treated F. diplosiphon, indicated increased lipid peroxidation and intracellular ROS in a dose-dependent manner. Both B481-WT and B481-SD strains treated with 3.2, 12.8, and 51.2 mg L⁻¹ nZVIs exhibited significant increases (p<0.01) in MDA content compared to the untreated control (FIG. 1). While the MDA content of the nZVI-treated B481-WT strain ranged from 86.73±9.7 to 165.50±5.8, that of the B481-SD strain ranged from 128.46±24 to 267.20±26.23 nmol mL⁻¹. However, we did not observe any significant differences in MDA concentration in both strains treated with 0.8, 1.6, and 102.4 mg L⁻¹ nZVIs. Cultures treated with 12.8 and 51.2 mg L−1 nZVIs exhibited significantly higher ROS compared to the untreated control, 1.6, and 3.2 mg L⁻¹ nZVIs (FIG. 1). Comparison of MDA content between strains in each treatment revealed significant differences from 1.6 to 102.4 mg L⁻¹ nZVIs (data not shown).

Increased ROS Detected by the 2′,7′-Dichlor-odihydrofluorescein Diacetate Assay. Intracellular ROS that oxidizes DCFH to highly fluorescent 2′,7′-dichlorofluorescein (DCF) ranged from 0.0710±0.001 to 0.1063±0.0006 in B481-WT, while that of the B481-SD ranged from 0.0647±0.0016 to 0.1±0.002 nmol mL⁻¹. Both strains treated with nZVIs ranging from 3.2 to 25.6 mg L⁻¹ exhibited significantly increased levels of ROS (p<0.001) compared to the untreated control (FIG. 2). While the wild-type strain exhibited significantly increased ROS levels when treated with 1.6-25.6 mg L⁻¹ (p<0.01) nZVIs, the B481-SD strain showed significantly higher levels of oxidative stress ranging from 3.2 to 25.6 mg L−¹ (p<0.01) nZVIs (FIG. 2). In addition, we observed significant differences between the B481-WT and B481-SD strains grown in 1.6, 6.4, 12.8, and 25.6 (p<0.05) mg L⁻¹ nZVIs (data not shown). The negative control of bare nanoparticles did not exhibit DCF signal interference.

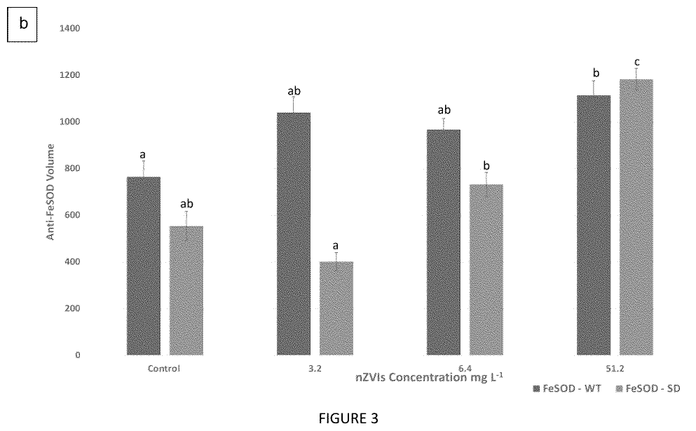

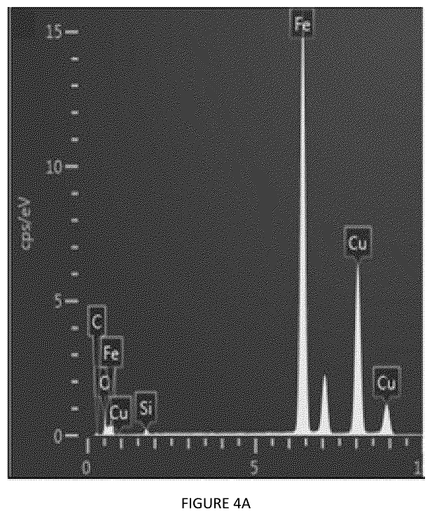

Immunodetection Reveals Increased SOD Levels in nZVI-Treated F. diplosiphon. SOD enzymes known for scavenging ROS in cells were quantified to determine oxidative stress in nZVI-treated F. diplosiphon. Densitometric analysis of anti-FeSOD revealed significantly higher FeSOD levels in both B481-WT (p<0.05) and B481-SD (p<0.001) strains treated with 51.2 mg L⁻¹ when compared to the control. We also observed significantly higher levels of FeSOD (731.97±89.6, p<0.01) in the B481-SD strain treated with 6.4 mg L⁻¹ nZVIs compared to 3.2 mg L⁻¹ (401.37±67.1, p<0.001); however, it was significantly lower than that in 51.2 mg L⁻¹ nZVIs. Additionally, the B481-SD strain treated with 51.2 mg L−1 exhibited significantly higher SOD levels compared to that of 3.2 mg L⁻¹ nZVIs. We did not observe significant differences in SOD levels of the B481-WT strain treated with 3.2 and 6.4 mg L−1 nZVIs compared to the untreated control (FIG. 3). Further, FeSOD activity was significantly higher in the B481-WT strain treated with 3.2 and 6.4 mg L⁻¹ nZVIs (p<0.05) compared to the B481-SD strain grown under similar concentrations (data not shown).

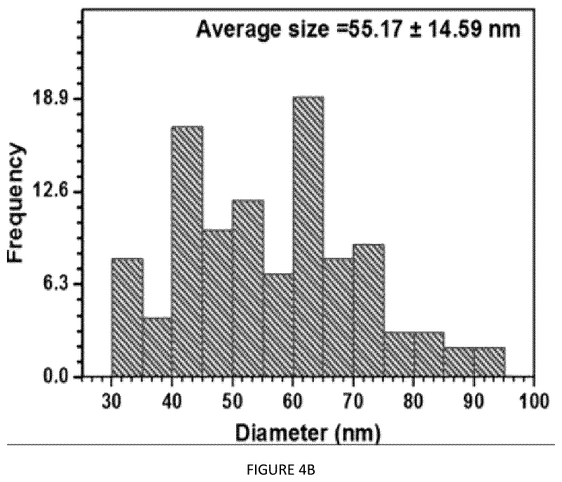

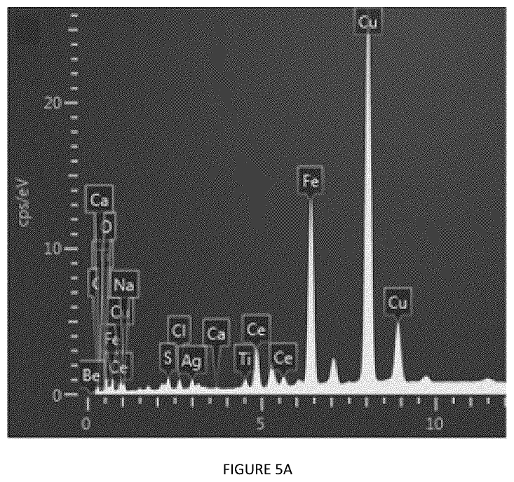

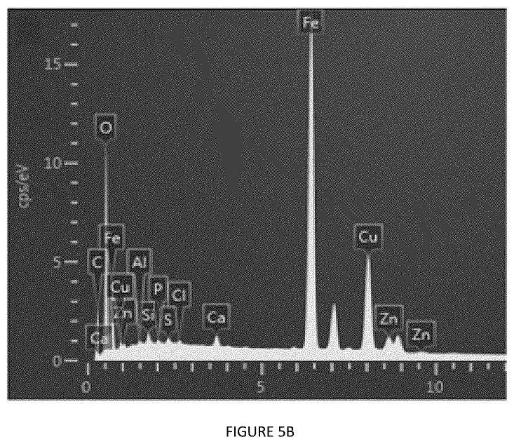

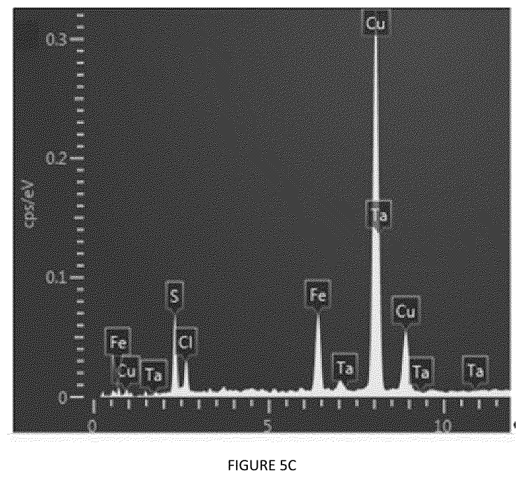

Particle Size Determination and F. diplosiphon-Nanoparticle Interaction. Optical imaging revealed adsorption of nZVIs on the surface of F. diplosiphon cells. TEM equipped with EDS confirmed the composition and distribution of nZVIs through elemental mapping and revealed the adsorption of nZVIs on F. diplosiphon cells. A strong iron peak in the EDS spectra confirmed the presence of elemental iron in nanofer 25 s nZVIs (FIG. 4a). As shown in the differential particle size histogram (FIG. 4b), nZVIs used in the study ranged from a size of 24.68 to 82.72 nm, with an average of 55.17 nm. Comparison of EDS spectra on days 4, 12, and 20 in both F. diplosiphon strains treated with 3.2 mg L⁻¹ nZVIs showed a gradual reduction of the iron signal over the period tested. While the spectral analysis of both B481-WT and B481-SD cultures treated with nZVIs indicated a strong signal for iron at day 4 (FIG. 5a) and day 12 (FIG. 5b), a significant decrease in the intensity of the iron peak was observed at day 20 (FIG. 5c). Control cultures that were not treated with nZVIs did not show the presence of the iron peak in EDS spectral analysis.

### CONCLUSIONS

The exposure of F. diplosiphon to nZVIs increases intracellular ROS production, leading to oxidative stress and membrane lipid peroxidation. In addition, the data show increased SOD enzyme activities as a defense mechanism to alleviate oxidative stress. TEM imaging with EDS analysis showed strong signals for iron at 4 and 12 days after treatment.

### REFERENCES

(1) Sunda, W. G.; Huntsman, S. A. High iron requirement for growth, photosynthesis, and low-light acclimation in the coastal cyanobacterium Synechococcus bacillaris. Front. Microbiol. 2015, 6, 561.
(2) Kranzler, C.; Rudolf, M.; Keren, N.; Schleiff, E. Iron in cyanobacteria. Adv. Bot. Res. 2013, 65, 57-105.
(3) Lea-Smith, D. J.; Bombelli, P.; Vasudevan, R.; Howe, C. J. Photosynthetic, respiratory and extracellular electron transport pathways in cyanobacteria. Biochim. Biophys. Acta 2016, 1857, 247-255.
(4) Sevcu, A.; EI-Temsah, Y. S.; Joner, E. J.; Cernik, M. Oxidative stress induced in microorganisms by zero-valent iron nanoparticles. Microb. Environ. 2011, 26, 271-281
(5) Padrova, K.; Lukavsky, J.; Nedbalova, L.; Cejkova, A.; Cajthaml, T.; Sigler, K.; Vitova, M.; Rezanka, T. Trace concentrations of iron nanoparticles cause overproduction of biomass and lipids during cultivation of cyanobacteria and microalgae. J. Appl. Phycol. 2015, 27, 1443-1451.
(6) Tran, D. L.; Le, V. H.; Linh Pham, H.; Nhung Hoang, T. M.; Quy Nguyen, T.; Luong, T. T.; Thu Ha, P.; Phuc Nguyen, X. Biomedical and environmental applications of magnetic nanoparticles. Adv. Nat. Sci. Nanosci. Nanotechnol. 2010, 1, 45013.
(7) Pan, F.; Zhong, X.; Xia, D.; Yin, X.; Li, F.; Zhao, D.; Ji, H.; Liu, W. Nanoscale zero-valent iron/persulfate enhanced upflow anaerobic sludge blanket reactor for dye removal: Insight into microbial metabolism and microbial community. Sci. Rep. 2017, 7, 44626.
(8) Haney, A. M.; Kehoe, D. M. Fremyella diplosiphon. Trends Microbiol. 2019, 27, 562-563.
(9) Tabatabai, B.; Fathabad, S. G.; Bonyi, E.; Rajini, S.; Aslan, K.; Sitther, V. Nanoparticle-mediated impact on growth and fatty acid methyl ester composition in the cyanobacterium Fremyella diplosiphon. BioEnergy Res. 2019, 12, 409-418.
(10) Fathabad, S. G.; Arumanayagam, A. S.; Tabatabai, B.; Chen, H.; Lu, J.; Sitther, V. Augmenting Fremyella diplosiphon cellular lipid content and unsaturated fatty acid methyl esters via sterol desaturase gene overexpression. Appl. Biochem. Biotechnol. 2019, 189, 1127-1140.
(11) Checa, J.; Aran, J. M. Reactive oxygen species: drivers of physiological and pathological processes. J. Inflammation Res. 2020, 13, 1057-1073.
(12) Fufezan, C.; Gross, C. M.; Sjödin, M.; Rutherford, A. W.; Krieger-Liszkay, A.; Kirilovsky, D. Influence of the redox potential of the primary quinone electron acceptor on photoinhibition in photosystem II. J. Biol. Chem. 2007, 282, 12492-12502.
(13) Pospíšil, P. Production of reactive oxygen species by photosystem II. Biochim. Biophys. Acta, Bioenerg. 2009, 1787, 1151-1160.
(14) Rajneesh; Pathak, J.; Chatterjee, A.; Singh, S. P.; Sinha, R. P. Detection of reactive oxygen species (ROS) in cyanobacteria using the oxidant-sensing probe 2′,7′-dichlorodihydrofluorescein diacetate (DCFH-DA). Bio-Protoc. 2017, 1857, 247-255.
(15) Yang, S.; Lian, G.; disease. ROS and diseases: role in metabolism and energy supply. Biochem 2020, 467, 1-12.
(16) Lee, J.-W.; Helmann, J. D. Functional specialization within the Fur family of metalloregulators. BioMetals 2007, 20, 485-499.
(17) Fathabad, S. G.; Tabatabai, B.; Walker, D. m.; Chen, H.; Lu, J.; Aslan, K.; Uddin, J.; Ghann, W.; Sitther, V.; Ghann, W.; Sitther, V. Impact of zero-valent iron nanoparticles on Fremyella diplosiphon transesterified lipids and fatty acid methyl esters. ACS Omega 2020, 5, 12166-12173.
(18) Latifi, A.; Ruiz, M.; Zhang, C.-C. Oxidative stress in cyanobacteria. FEMS Microbiol. Rev. 2009, 33, 258-278.
(19) Padmapriya, V.; Anand, N. The influence of metals on the antioxidant enzyme, superoxide dismutase, present in the cyanobacterium, Anabaena variabilis KUTZ. ARPN J. Agric. Biol. Sci. 2010, 5, 4-9.
(20) Lei, C.; Zhang, L.; Yang, K.; Zhu, L.; Lin, D. Toxicity of iron-based nanoparticles to green algae: Effects of particle size, crystal phase, oxidation state and environmental aging. Environ. Pollut. 2016, 218, 505-512.
(21) Kunui, K.; Minj, R. A.; Singh, S. S.; Vishwavidyalaya, G. G.; Biology, S. Iron transportation and its impact on nitrate assimilation in the cyanobacterium Anabaena sphaerica: A promising bioaccumu-lator of iron. Plant Arch. 2020, 20, 3701-3709.
(22) Priya, B.; Premanandh, J.; Dhanalakshmi, R. T.; Seethalakshmi, T.; Uma, L.; Prabaharan, D.; Subramanian, G. Comparative analysis of cyanobacterial superoxide dismutases to discriminate canonical forms. BMC Genom. 2007, 8, 435.
(23) Li, T.; Huang, X.; Zhou, R.; Liu, Y.; Li, B.; Nomura, C.; Zhao, J. Differential expression and localization of Mn and Fe superoxide dismutases in the heterocystous cyanobacterium Anabaena sp. strain PCC 7120. J. Bacteriol. 2002, 184, 5096-5103.
(24) Ismaiel, M. M. S.; El-Ayouty, Y. M.; Loewen, P. C.; Piercey-Normore, M. D. Characterization of the iron-containing superoxide dismutase and its response to stress in cyanobacterium Spirulina (Arthrospira) platensis. J. Appl. Phycol. 2014, 26, 1649-1658.
(25) Hu, J.; Li, M.; Gao, Q.; Niou, C.; Kary Chien, W. T. Energy dispersive spectrum (EDS) study of copper grid effect on semiconductor failure analysis. ASM Intern. 2006, 297-299.
(26) Taghizadeh, S.-M.; Berenjian, A.; Chew, K. W.; Show, P. L.; Mohd Zaid, H. F.; Ramezani, H.; Ghasemi, Y.; Raee, M. J.; Ebrahiminezhad, A. Impact of magnetic immobilization on the cell physiology of green unicellular algae Chlorella vulgaris. Bioengineered 2020, 11, 141-153.
(27) Saxena, P.; Sangela, V.; Harish. Toxicity evaluation of iron oxide nanoparticles and accumulation by microalgae Coelastrella terrestris. Environ. Sci. Pollut. Res. 2020, 27, 19650-19660.
(28) Bhandari, R.; Sharma, P. K. High-light-induced changes on photosynthesis, pigments, sugars, lipids and antioxidant enzymes in freshwater (Nostoc spongiaeforme) and marine (Phormidium corium) cyanobacteria. Photochem. Photobiol. 2006, 82, 702.
(29) Pattanaik, B.; Busch, A. W. U.; Chen, J.; Montgomery, B. L. Responses to iron limitation are impacted by light quality and regulated by RcaE in the chromatically acclimating cyanobacterium Fremyella diplosiphon. Microbiol 2014, 160, 992-1005.
(30) Smith, P. K.; Krohn, R. I.; Hermanson, G. T.; Mallia, A. K.; Gartner, F. H.; Provenzano, M. D.; Fujimoto, E. K.; Goeke, N. M.; Olson, B. J.; Klenk, D. C. Measurement of protein using bicinchoninic acid. Anal. Biochem. 1985, 150, 76-85.
(31) Laemmli, U. K. Cleavage of structural proteins during the assembly of the head of bacteriophage T4. Nature 1970, 227, 680-685.
(32) O'Farrell, P. H. High-resolution two-dimensional electrophoresis of proteins. J. Biol. Chem. 1975, 250, 4007-4021.

1. A method for detecting and/or quantifying nZVI-mediated oxidative stress in cyanobacterium, the method comprising:
a. growing a first culture of said cyanobacterium in the presence of a concentration of zero-valent iron nanoparticles
b. growing a control culture of said cyanobacterium in the absence of zero-valent iron nanoparticles;
c. assaying said first culture and said control culture for lipid peroxidation;
d. comparing a level of lipid peroxidation in said first culture and a level of lipid peroxidation in said control culture;
e. making a determination that said concentration of zero-valent iron nanoparticles in said first culture of said cyanobacterium is causing oxidative stress in said first culture of said cyanobacterium when said level of lipid peroxidation in said first culture of said cyanobacterium is statistically higher than said level of lipid peroxidation in said control culture of said cyanobacterium.

2. The method of claim 1, wherein said assaying of said first culture and said control culture of lipid peroxidation comprises a determination of malondialdehyde content in said first culture and said control culture.

3. The method of claim 1, wherein said cyanobacterium is F. diplosiphon.

4. The method of claim 1, wherein said cyanobacterium is B481-WT F. diplosiphon.

5. The method of claim 1, wherein said cyanobacterium is B481-SD F. diplosiphon.

6. The method according to claim 1, further comprising carrying out steps a and c-e with a second culture of said cyanobacterium in the presence of a second concentration of zero-valent iron nanoparticles.

7. A method for detecting and/or quantifying nZVI-mediated oxidative stress in cyanobacterium, the method comprising:
a. growing a first culture of said cyanobacterium in the presence of a concentration of zero-valent iron nanoparticles
b. growing a control culture of said cyanobacterium in the absence of zero-valent iron nanoparticles;
c. adding a fluorometric probe to each of said first culture and said control culture;
d. measuring a fluorescence of said first culture and said control culture
e. comparing a level of fluorescence of said first culture and said control culture;
f. making a determination that said concentration of zero-valent iron nanoparticles in said first culture of said cyanobacterium is causing oxidative stress in said first culture of said cyanobacterium when said level of fluorescence of said first culture of said cyanobacterium is statistically higher than said level of fluorescence in said control culture of said cyanobacterium.

8. The method of claim 7, wherein said fluorometric probe is 2′,7′-dichlorodihydrofluorescein diacetate fluorometric probe.

9. The method of claim 7, wherein said cyanobacterium is F. diplosiphon.

10. The method of claim 7, wherein said cyanobacterium is B481-WT F. diplosiphon.

11. The method of claim 7, wherein said cyanobacterium is B481-SD F. diplosiphon.

12. The method according to claim 7, further comprising carrying out steps a and c-f with a second culture of said cyanobacterium in the presence of a second concentration of zero-valent iron nanoparticles.

13. A method for detecting and/or quantifying nZVI-mediated oxidative stress in cyanobacterium, the method comprising:
a. growing a first culture of said cyanobacterium in the presence of a concentration of zero-valent iron nanoparticles
b. growing a control culture of said cyanobacterium in the absence of zero-valent iron nanoparticles;
c. carrying out a densitometric analysis of superoxide dismutase (“SOD”) in said first culture and said control culture
d. comparing a level of SOD in said first culture and said control culture;
e. making a determination that said concentration of zero-valent iron nanoparticles in said first culture of said cyanobacterium is causing oxidative stress in said first culture of said cyanobacterium when said level of SOD in said first culture of said cyanobacterium is statistically higher than said level of SOD in said control culture of said cyanobacterium.

14. The method of claim 13, wherein said SOD is Fe-SOD.

15. The method of claim 13, wherein said cyanobacterium is F. diplosiphon.

16. The method of claim 13, wherein said cyanobacterium is B481-WT F. diplosiphon.

17. The method of claim 13, wherein said cyanobacterium is B481-SD F. diplosiphon.

18. The method according to claim 13, further comprising carrying out steps a and c-e with a second culture of said cyanobacterium in the presence of a second concentration of zero-valent iron nanoparticles.

19. A method for detecting and/or quantifying cellular update and/or surface adsorption of nZVI by cyanobacterium, the method comprising:
a. growing a first culture of said cyanobacterium in the presence of a concentration of zero-valent iron nanoparticles
b. growing a control culture of said cyanobacterium in the absence of zero-valent iron nanoparticles;
c. conducting transmission electron microscopy (TEM) and energy-dispersive X-ray spectroscopy (EDS) analysis on each of said first culture and said control culture over a period of days;
d. analyzing changes in TEM and EDS for said first culture over time, and comparing said changes to TEM and EDS for said control culture over time.

20. The method of claim 16, wherein said cyanobacterium is F. diplosiphon.

21. The method of claim 16, wherein said cyanobacterium is B481-WT F. diplosiphon.

22. The method of claim 16, wherein said cyanobacterium is B481-SD F. diplosiphon.

23. The method according to claim 16, further comprising carrying out steps a and c and d with a second culture of said cyanobacterium in the presence of a second concentration of zero-valent iron nanoparticles.
